(19) United States
(12) Patent Application Publication
(10) Pub. No.: US 2025/0241882 A1
(43) Pub. Date: Jul. 31, 2025

(54) COMPOSITION FOR TREATMENT OF THE SKIN

(71) Applicant: DR. KURT WOLFF GMBH & CO. KG, Bielefeld (DE)

(72) Inventors: Erik SCHULZE ZUR WIESCHE, Bielefeld (DE); Maike BECKER, Bielefeld (DE); Jörn Michael VÖLKER

(73) Assignee: DR. KURT WOLFF GMBH & CO. KG, Bielefeld (DE)

(21) Appl. No.: 18/854,001

(22) PCT Filed: Apr. 6, 2023
(86) PCT No.: PCT/EP2023/059204

§ 371 Date: Oct. 3, 2024

(30) Foreign Application Priority Data
Apr. 6, 2022 (DE) 10 2022 108 329.2

(51) Int. Cl. A61K 31/197; A61K 8/365; A61K 8/44; A61K 9/00; A61K 31/194; A61Q 19/00

(52) U.S. Cl. CPC A61K 31/197; A61K 8/365; A61K 8/44; A61K 9/0014; A61K 31/194; A61Q 19/00; A61K 2800/10

(57) ABSTRACT

The present invention relates to a topical composition containing dimethylglycine and/or a salt of dimethylglycine and at least one carboxylic acid in a weight ratio of 1:0.1 to 1:10. The invention also relates to the medical and/or cosmetic use of this composition for treating the skin.

The present invention relates to a composition containing dimethylglycine and/or a salt of dimethylglycine. Furthermore, the present invention relates to the (medical and/or cosmetic) use of this composition for treatment of the skin.

The skin is the largest organ in humans. Among its many functions (for example, heat regulation and as a sensory organ), the barrier function and protective function, which prevent the skin (and thus ultimately the entire organism) from drying out, are probably the most important. At the same time, the skin acts as protection against the entrance and absorption of external substances. In particular, harmful UV rays, microbiological attacks and the penetration of pollutants that are foreign to the skin should be mentioned here. This barrier function is achieved by the epidermis (top layer of the skin), which is the outermost layer and forms the actual protective cover with respect to the environment. Particular attention is paid to the outer horny layer (stratum corneum)—a resistant cell layer whose individual horny cells are connected by the lipid barrier. The lipid barrier protects the skin from drying out and maintains its water-retaining state. The natural pH of the skin's surface is usually between pH 4-7. At about one tenth of the total thickness of the skin, the epidermis is simultaneously the thinnest layer of the skin. In addition to the epidermis, this layer also includes the underlying mesodermal connective tissue of the corium (dermis, subcutaneous tissue). Both layers are intimately interlocked by a system of papilla- or strip-shaped corium protrusions and epidermal pegs that extend deeply therebetween. The epidermis also derives its nutrition from the corium, which has a rich blood supply. Pigment cells in the corium provide the pigments that give the skin its color, and the corium papillae contain the sensory receptors for the sensory functions of the skin regarding touch, pain and temperature sensation.

The superficial cell layers of the epidermis are subject to constant wear. In addition, the epidermis can react very sensitively to external influences with irritation, redness, itching, flaking and cracking. In addition, it is constantly exposed to the risk of inflammation and injuries (wounds). In order to thus ensure optimal protection, the epidermis therefore completely renews itself within approximately 30 days. This epidermal renewal results from the continuous replacement of a basal germ layer that actively divides throughout its life.

Cosmetic and dermatological preparations for skin care are known. Such compositions are thus based on O/W or W/O emulsions, which are based, for example, on a mixture of long-chain fatty acids, mono- and/or diglycerides of fatty acids, ethoxylated fatty acid esters, non-polar lipids, fatty alcohols, lipophilic thickening agents, hydrogenated polyisobutenes and polar lipids. In particular, to improve the skin barrier of the epidermis, many cosmetic compositions are now available, via which fatty substances are usually supplied to the skin from the outside. However, such compositions have a number of disadvantages. Penetration into the skin is often rather low, and so the skin can feel unpleasantly greasy. In most cases, the fatty substances are also foreign to the body and therefore cannot satisfactorily regenerate a weakened skin barrier. With increasing age, the activity of keratinocytes also decreases, meaning that cell division and skin regeneration and thus the formation of the body's own skin barrier decreases.

There is therefore a need for improved pharmaceutical and cosmetic treatment methods, in particular a need for compositions that both strengthen the skin's protective and barrier function and at the same time improve the skin's feel and appearance, whereby these compositions are not supposed to display any side effects, or display only negligible side effects. In particular, there is a need for compositions that support the renewal of the epidermis and compensate for impairments of the skin caused by previous stresses, such as, inter alia, poorly healing wounds, irritations, inflammations and others.

Proceeding therefrom, the object of the present invention was to provide a well-tolerated composition for treating the skin, in particular for strengthening the skin's protective and barrier function. Furthermore, this composition should be applicable topically and overcome the disadvantages of the compositions known from the prior art.

In particular, the object of the present invention was to provide an effective and well-tolerated composition that supports the regeneration of the epidermis and has a positive effect on wound healing. At the same time, the composition should be easy to process.

This object was surprisingly achieved by the composition according to claim 1 and the use thereof according to claim 8. Preferred embodiments can be found in the dependent claims. According to the invention, the object is achieved by providing a topical composition which comprises dimethylglycine and/or a salt of dimethylglycine and at least one carboxylic acid in a specific weight ratio.

It has surprisingly been found that the topical composition according to the invention comprising a combination of dimethylglycine and/or a salt of dimethylglycine and at least one carboxylic acid is incredibly effective at strengthening the skin's protective and barrier function. The composition activates the skin and significantly improves the nutrient and oxygen supply to the skin and hair roots. In addition, it is medically and cosmetically extremely well tolerated on the skin. Without being bound by any theory, it is believed that the topical composition according to the invention surprisingly increases the activity of the keratinocyte cells and thus causes or positively influences the strengthening of the skin barrier. Furthermore, it has surprisingly been found that, through the combined presence of a) dimethylglycine and/or a salt of dimethylglycine and b) a carboxylic acid according to the invention, the composition increases skin moisture, increases the osmotic pressure of the composition (i.e., the total number of dissolved chemical units in the composition), accelerates and generally improves the release of the active ingredient (i.e., dimethylglycine and/or a salt of dimethylglycine) from the composition and accelerates and generally improves the penetration of the active ingredient into and through the skin.

N, N-dimethylglycine occurs naturally in animals and plants and is an intermediate in the metabolism of choline. N,N-dimethylglycine is a weak acid according to the chemical definition of the acid dissociation constant and forms an alkaline solution in aqueous systems. N,N-dimethylglycine, also (dimethylamino) acetic acid, is represented by the following chemical formula 1:

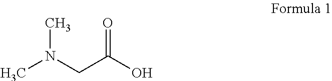

The invention relates not only to the use of dimethylglycine, but also to its salts, solvates and hydrates. These are preferably pharmaceutically or cosmetically acceptable salts of dimethylglycine. The salt is particularly preferably a water-soluble salt with a solubility in water of at least 10 g/L at 20° C. Betaine structures and betaine-like structures, i.e., compounds with fully alkylated ammonium units (specifically, any compounds with 4-fold C-substituted N atoms), are explicitly not intended to be included in this formula.

In a preferred embodiment, the salt of dimethylglycine is an alkali, alkaline earth or ammonium salt of dimethylglycine.

Examples include sodium, potassium, calcium, magnesium and ammonium salts. In ammonium salts, the ammonium cation carries one to four alkyl groups, each independently containing 1 to 4 carbon atoms. Preferred are the sodium and potassium salt of dimethylglycine, in particular the sodium salt of dimethylglycine, specifically sodium-N,N-dimethylglycinate.

In an alternatively preferred embodiment, the salt of dimethylglycine may be the salt of an inorganic and/or organic acid with dimethylglycine.

Examples of salts of dimethylglycine with an inorganic acid are the hydrochloride, hydrobromide, hydroiodide, hydrogen sulfate, sulfate, hydrogen sulfite, sulfite, hydrogen carbonate, carbonate, monophosphate, diphosphate and triphosphate of dimethylglycine, and mixtures thereof. Particularly preferred is the hydrochloride of dimethylglycine.

Examples of salts of dimethylglycine with an organic acid are the acetate, lactate, citrate, succinate, fumarate, maleate and benzoate of dimethylglycine, and mixtures thereof.

The use of dimethylglycine and/or a salt of dimethylglycine in the topical composition according to the invention supports the regeneration of the epidermis and wound healing, smoothes the skin, strengthens the skin's protective and barrier function, and has a positive effect on skin with inflammatory conditions. In addition, it was found that the activity of keratinocytes was also significantly increased by dimethylglycine and/or a salt of dimethylglycine. According to the invention, it is used both for the prevention and for the treatment of disorders caused by the lack of regeneration of the epidermis.

It is believed that dimethylglycine and/or a salt of dimethylglycine according to the invention improves cell activity and oxygen turnover in the keratinocytes and thus also promotes cell activity in the skin. According to the invention, it thus achieves a significant skin-strengthening effect, particularly in the treatment of skin that is stressed or weakened by everyday life or age. In addition to dimethylglycine and/or salts of dimethylglycine, the compositions according to the invention contain at least one carboxylic acid. Preferably, the carboxylic acid is selected from the group consisting of adipic acid, ascorbic acid, lactic acid, acetic acid, propanoic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methane sulfonic acid, ethane sulfonic acid, p-toluene sulfonic acid, salicylic acid, azelaic acid, cinnamic acid, formic acid, (meth)acrylic acid, nicotinic acid and mixtures thereof. Very particularly preferred are lactic acid, citric acid, salicylic acid, malic acid, fumaric acid and tartaric acid, as well as mixtures of two or more of these carboxylic acids.

The at least one carboxylic acid does not only set a pH value of the composition that is adjusted to the skin, but also surprisingly synergistically enhances the above-mentioned effects of dimethylglycine and/or the salts of dimethylglycine on the skin. This results in an increase in the activity of keratinocyte cells and the strengthening of the skin barrier, the increase in skin moisture and the osmotic pressure of the composition, the improvement and acceleration of the release of the active ingredient from the composition, and an acceleration and improvement of the penetration into and through the skin.

The weight ratio of dimethylglycine or a salt of dimethylglycine to carboxylic acid is 1:0.1 to 1:10. Preferably, the ratio ranges from 1:0.5 to 1:5, particularly preferably from 1:0.6 to 1:2, most preferably from 1:0.7 to 1:1.5. Explicitly usable preferred weight ratios are: 1:0.50; 1:0.60; 1:0.70; 1:0.75; 1:0.80; 1:0.85; 1:0.90; 1:0.95; 1:1; 1:1.05; 1:1.10; 1:1.20; 1:1.25; 1:1.30; 1:1.40; 1:1.50; 1:1.60; 1:1.70; 1:1.80 and 1:1.90. With these weight ratios mentioned, the effects according to the invention are achieved particularly effectively.

The pH of the topical composition is preferably 3.0 to 8.0, preferably 3.0 to 5.9, more preferably 3.5 to 5.4, and particularly preferably 4.0 to 5.0 (measured at 21° C. using a pH meter, Mettler-Toledo SevenCompact S220). Within this range, the compositions according to the invention are not only particularly chemically, physically and microbiologically stable, but are also medically and cosmetically extremely well tolerated by the skin. Particularly suitable pH values for the composition according to the invention are: 3.5; 3.6; 3.7; 3.8; 3.9; 4.0; 4.1; 4.2; 4.3; 4.4; 4.5; 4.6; 4.7; 4.8; 4.9; 5.0; 5.1; 5.2; 5.3 and 5.4. The pH of the composition is preferably adjusted using one or more pH modifiers. In addition to the carboxylic acids used according to the invention, suitable pH modifiers are other acids, bases and/or buffer systems which stabilize or influence the pH value of the composition. Typical pH modifiers according to the present invention are, in addition to the carboxylic acids mentioned, also their corresponding salts, as well as sodium alginate, polyacrylic acid, sodium carbonate and sodium bicarbonate. In the context of pH modifiers, the term “salt” refers to alkali metal salts or alkaline earth metal salts unless stated otherwise.

The composition according to the invention contains dimethylglycine and/or a salt of dimethylglycine preferably in a proportion of 0.00001 wt. % to 25.0 wt. %, based on the total weight of the composition. In a preferred embodiment, the composition according to the invention contains dimethylglycine and/or a salt of dimethylglycine in a proportion of 0.001 wt. % to 10.0 wt. %, more preferably from 0.01 wt. % to 8.0 wt. %, more preferably from 0.1 wt. % to 6.0 wt. %, even more preferably from 0.3 wt. % to 5.0 wt. %, in particular from 0.5 wt. % to 3.0 wt. %, in each case based on the total weight of the composition. In a preferred embodiment of the invention, the composition according to the invention can contain 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1.0 wt. %, 1.1 wt. %, 1.2 wt. %, 1.3 wt. %, 1.4 wt. %, 1.5 wt. %, 2.0 wt. % or 2.5 wt. % dimethylglycine and/or a salt of dimethylglycine, each based on the total weight of the composition. The composition according to the invention preferably contains dimethylglycine and/or a salt of dimethylglycine as a pure chemical substance, including the solvates and hydrates (e.g., the dihydrate of sodium dimethylglycinate), since this can increase the purity of the composition and reduce the occurrence of undesirable side effects. For this reason, the composition according to the invention preferably contains the chemical derivatives of dimethylglycine selected from methylglycine, trimethylglycine, (2-hydroxyethyl)trimethyl ammonium, and trimethyl-hydroxybutyrobetaine, in concentrations of less than 0.01 wt. %, based on the total weight of the composition. Particularly preferably, the compositions according to the invention are completely free of these derivatives.

Preferably, the composition according to the invention does not contain the following combinations:
a) 1.0 wt. % Na-dimethylglycinate and 0.2 wt. % citric acid;
b) 0.4 wt. % Na-dimethylglycinate and 0.2 wt. % citric acid;
c) 0.5 wt. % Na-dimethylglycinate and 0.1 wt. % lactic acid;
d) 0.4 wt. % Na-dimethylglycinate and citric acid (q.s. pH 5.5);
e) 0.4 wt. % Na-dimethylglycinate and glycolic acid (q.s. pH 4.0);
f) 0.4 wt. % Na-dimethylglycinate and lactic acid (q.s. for pH 5.9);
g) 1.0 wt. % dimethylglycine HCl and 0.2 wt. % citric acid;
h) 0.5 wt. % dimethylglycine HCl and 0.1 wt. % lactic acid.

In a preferred embodiment, the composition according to the invention contains at least one further active ingredient, wherein the at least one further active ingredient is selected from caffeine, menthol, biotin, zinc PCA, niacinamide, panthenol, ectoine, ubiquinone-10, taurine, pantolactone, echinacea, tocopheryl acetate and combinations thereof. Preferably, the active ingredient or the combination of active ingredients does not comprise the compounds methylmethionine, glycerophosphocholine, phosphatidylcholine, N-acylethanolamine, and/or N-acylphosphatidylethanolamine, e.g., with a C1-C22 acyl residue.

Alternatively, the topical composition according to the invention currently does not contain caffeine. This embodiment is also referred to as a (completely) caffeine-free topical composition or as a topical composition according to the invention devoid of caffeine, to which all other preferred features of the invention described herein apply, apart from said caffeine. Furthermore, the combination of dimethylglycine or one of the salts of dimethylglycine and one or more of the following active ingredients is preferred according to the invention.

Menthol is a monocyclic monoterpene alcohol and can be added to the composition according to the invention as an active blood circulation stimulating ingredient. Menthol can also provide a refreshing sensory stimulation of the skin.

Biotin, also known as vitamin B₇ or vitamin H, is a water-soluble vitamin from the B-complex. According to the invention, biotin can strengthen the skin.

Zinc PCA is the zinc salt of L-pyrrolidone carboxylate and can be added to the composition according to the invention as a substance having an antimicrobial effect.

Niacinamide (also nicotinamide) is the amide of nicotinic acid and is also called vitamin B₃. In addition to other properties such as reducing oxidative stress, according to the invention niacinamide has a skin-stimulating effect.

Panthenol is a provitamin which is converted into pantothenic acid (vitamin B₅) in the body. The latter is part of co-enzyme A and is therefore important for skin metabolism. According to the invention, when panthenol is applied, skin elasticity and moisture are further improved. In addition, itching and inflammation are relieved, and wound healing is promoted.

Ectoin is a cyclic amino acid and exists in aqueous solution as a zwitterion stabilized by mesomerism. Ectoin has a moisturizing effect and, according to the invention, further stabilizes the skin's natural structure. Ectoin has also been shown to protect against UV radiation and may be helpful in the treatment of inflammatory diseases.

Ubiquinone-10 (Q10 or co-enzyme Q₁₀) is a quinone derivative. Q10, which belongs to the ubiquinone pool, is considered an antioxidant and, according to the invention, has a stabilizing effect on the skin.

According to the invention, taurine or 2-aminoethane sulfonic acid also acts as an antioxidant and further stabilizes the skin.

Pantolactone comes from the group of substituted lactones and, according to the invention, further stimulates the skin's growth factors.

According to the invention, echinacea has a calming effect on the skin and relieves itching and tension. Echinacea can also stimulate blood circulation in the skin.

Tocopheryl acetate exhibits antioxidant properties and, according to the invention, further stabilizes the skin.

In a preferred embodiment of the invention, the composition according to the invention contains at least one further active ingredient selected from menthol, biotin, zinc PCA, niacinamide, panthenol, ectoin, ubiquinone, taurine, pantolactone, echinacea, tocopheryl acetate and combinations thereof, each in a proportion of 0.001 wt. % to 10.0 wt. %, more preferably 0.005 wt. % to 7.50 wt. %, even more preferably from 0.01 wt. % to 5.0 wt. %, in particular from 0.1 wt. % to 3.0 wt. %, based on the total weight of the composition.

The composition according to the invention is preferably in the form of an emulsion or a gel (gel formulation) or as a mixture of an emulsion and a gel (emulgel). Suitable emulsions comprise at least an aqueous phase and an oil phase (lipophilic phase) as well as an emulsifier system. The lipophilic phase preferably amounts to 25 to 70 wt. % of the composition (emulsion), particularly preferably 35 to 60 wt. %, and very particularly preferably 40 to 55 wt. %. The aqueous phase preferably amounts to 20 to 65 wt. % of the composition (emulsion), particularly preferably 30 to 55 wt. %, and very particularly preferably 35 to 50 wt. %. The emulsifier system preferably amounts to 2 to 10 wt. %, particularly preferably 4 to 7 wt. %, of the composition (emulsion). According to the invention, the type of emulsion is not critical. W/O, O/W, W/O/W and O/W/O emulsions are suitable according to the invention. Emulsions suitable according to the invention are known to those skilled in the art (e.g., from DE102012002950A1 or DE102010029628A1).

However, this also means that the emulsion according to the invention preferably does not comprise any further carrier substances. Preferably, the composition according to the invention does not contain a lamellar structure (in particular no vesicles with a lamellar double membrane structure and/or liposomes). It preferably does not contain any substance(s) that form lamellar double membrane structures either.

Suitable gel formulations of the invention contain a gelling agent (also called a gellant). In general, all gelling agents known in the pharmaceutical and/or cosmetic field for the preparation of stable gel formulations can be used in the context of the invention. Preferably, hydrophilic gelling agents are used. Examples of suitable gelling agents are natural gelling agents such as pectin, agarose, gelatin and casein or modified natural gelling agents such as cellulose derivatives including methylcellulose, hydroxymethylcellulose, hydroxyethylcellulose (HEC), hydroxymethylpropylcellulose (HPMC) and carboxymethylcellulose, or fully synthetic gelling agents such as polyvinyl alcohols, poly(meth)acrylic acids, polyacrylamide, polyvinylpyrrolidone, polypropylene glycol, polyethylene glycol and poloxamers. Cellulose derivatives and poloxamers are preferred because of the higher stability and the appropriate viscosity of the gels obtained. Poloxamers are most preferred for reasons of gel stability and because of their function as solubilizers. Suitable poloxamers are Poloxamer 407 (e.g., trade name Pluronic F 127; Kolliphor P 407), or Poloxamer 188 (e.g., trade name Pluronic F 68; Kolliphor P 188).

Suitable gel formulations contain up to 20 wt. % lipophilic components, preferably 10 to 20 wt. %, based on the gel formulation. Gel formulations suitable according to the invention are known to the person skilled in the art.

Suitable mixed formulations consisting of an emulsion and gel (emulgels) which can be used according to the invention are also known to the person skilled in the art.

Preferably, the composition (in particular the emulsion or gel) has a viscosity of 800 to 20,000 mPa·s. Particularly preferred lower limits of this range are 1,000 mPa·s, 2,000 mPa·s, 3,000 mPa·s, 4,000 mPa·s, 5,000 mPa·s, 6,000 mPa·s, 7,000 mPa·s, 8,000 mPa·s, 9,000 mPa·s, and 10,000 mPa·s. Particularly preferred upper limits of this range are 19,000 mPa·s, 18,000 mPa·s, 17,000 mPa·s, 16,000 mPa·s, 15,000 mPa·s, 14,000 mPa·s, 13,000 mPa·s, and 12,000 mPa·s. Very particularly preferably, the range extends from above 6,000 to 20,000 mPa·s, preferably from 6,500 to 20,000 mPa·s and particularly preferably from 7,500 to 15,000 mPa·s. The viscosity values are each measured in accordance with DIN 53019-1:2008-09 using the Haake RheoStressl rheometer (ThermoFisher Scientific) at 20° C. and a shear rate of 10/s in plate-plate geometry (PP60 Ti rotating body). In the above-mentioned viscosity ranges according to the invention, not only is the release of the active ingredient (i.e., dimethylglycine and/or a salt of dimethylglycine) from the composition particularly significantly accelerated and improved on the whole, but also the penetration of the active ingredient into and through the skin is significantly faster and more complete.

In one embodiment, the composition comprises a surfactant. The surfactant can be an anionic, non-ionic, cationic or zwitterionic surfactant. Preferably, the surfactant is an anionic or non-ionic surfactant, in particular an anionic or non-ionic surfactant that is as mild as possible (i.e., particularly compatible with the skin). According to the invention, non-ionic surfactants are used in particular because of their very good emulsification properties and their excellent skin care properties. Anionic surfactants are preferred because they have a particularly high cleaning performance.

The composition according to the invention preferably contains surfactants in an amount of 2 to 40 wt. %, in particular of 5 to 30 wt. %, preferably of 7 to 20 wt. %, particularly preferably of 10 to 17 wt. %, in each case based on the total weight of the composition. Suitable amounts of surfactant are: 8 wt. %; 9 wt. %; 10 wt. %; 11 wt. %; 12 wt. %; 13 wt. %; 14 wt. %; 15 wt. %; 16 wt. %; 17 wt. %; 18 wt. %; 19 wt. %; 20 wt. %; 21 wt. %; 22 wt. %; 23 wt. %; 24 wt. %; 25 wt. %, in each case based on the total weight of the composition. Particularly preferably, the topical composition according to the invention contains one or more anionic surfactants in an amount of 0.1 to 20 wt. %, preferably 1 to 17 wt. % and particularly preferably 5 to 15 wt. %, in each case based on the total weight of the composition. Suitable amounts of anionic surfactant are: 1 wt. %; 2 wt. %; 3 wt. %; 4 wt. %; 5 wt. %; 6 wt. %; 7 wt. %; 8 wt. %; 9 wt. %; 10 wt. %; 11 wt. %; 12 wt. %; 13 wt. %; 14 wt. %; 15 wt. %; 16 wt. %; 17 wt. %; 18 wt. %, 19 wt. %, 20 wt. %, in each case based on the total weight of the composition. In these quantities, the surfactants have a particularly high cleaning performance and are extremely well tolerated by the skin.

The surfactants of the present invention are described, among others, in the book “Surfactants and interfacial phenomena,” by Milton Rosen and Joy Kunjappu, John Wiley & Sons, Inc.-Verlag, 2012, 4th edition.

In a preferred embodiment, the surfactant is an anionic surfactant selected from alkyl sulfonates, alkyl sulfates, alkyl ether sulfates, alkyl phosphates, alkyl sarcosinates, alkyl taurates, amino acid surfactants and mixtures thereof. Particularly preferably, the surfactant is selected from alkyl sulfates, alkyl sarcosinates, alkyl taurates, alkyl glutamate such as sodium cocoyl glutamate/disodium cocoyl glutamate, alkyl glycinate, alkyl alaninate, such as sodium cocoyl alaninate and mixtures thereof. Also preferred because of their cleaning performance are fatty alcohol polyglycerol ether sulfates, monoglyceride sulfates, mono- and/or dialkyl sulfosuccinates, fatty acid isethionates, and α-olefin sulfonates.

Alkyl sulfates have the generic formula ROSO₃M, alkyl sarcosinates have the generic formula RC(O)N(CH₃)CH₂CO₂M, and alkyl taurates have the generic formula RC(O)N(CH₃)CH₂CH₂SO₃M, where R is a C₄-C₂₆ alkyl or C₄-C₂₆ alkenyl in each case, and M is a water-soluble cation such as ammonium, sodium or potassium. Preferably, M is a sodium cation. Preferably, R is a C₁₂-C₁₆ alkyl or a C₁₂-C₁₈ alkyl.

In one embodiment, the surfactant is a non-ionic surfactant (also called a non-ionic emulsifier). Non-limiting examples include glycerol fatty acid esters, polyoxyethylene ethers of one or more fatty alcohols, alkoxylated fatty acid alkyl esters, polyglycerol ethers of fatty alcohols, polyglycerol esters of fatty acids, polyethylene glycol and/or polypropylene glycol ethers, fatty acid amides, alkylphenol polyglycol ethers, amine oxides, and alkyl polyglucosides.

In one embodiment, the surfactant is selected from the group of glycerol fatty acid esters, polyoxyethylene ethers of one or more fatty alcohols, polyglycerol ethers of fatty alcohols, polyglycerol esters of fatty acids and mixtures thereof.

In the present invention, the term “glycerol fatty acid ester” refers to a glycerol monofatty acid ester or a glycerol difatty acid ester. Glycerol difatty acid esters have the formula R³—COO—(CH₂CH(OH)CH₂)—OOR⁴ or R³—COO—(CH₂CH(OOR⁴)CH₂)—OH. Glycerol monofatty acid esters have the formula R³—COO—(CH₂CH(OH)CH₂)—OH or HO—(CH₂CH(OOR³)CH₂)—OH. R³ and R⁴ are independently selected from C₆-C₂₈ alkyl and C₆-C₂₈ alkenyl. Glycerol monofatty acid esters contain a glycerol group which is bound to a single fatty acid via an ester bond. Examples are glycerol monostearate, glycerol monobehenate, glycerol monocaprylate, glycerol monocaprate and glycerol monolaurate.

Polyoxyethylene ethers are compounds of the formula R⁵(OC₂H₃)ₙOH, where R⁵ is selected from C₆-C₂₈ alkyl, C₆-C₂₈ alkenyl, substituted and unsubstituted phenoxy groups; and n is an integer greater than 1. Preferably, the polyoxyethylene ether of one or more fatty alcohols is selected from the group of steareth-2, steareth-21, macrogol-cetostearyl ether 12, ceteareth-25, macrogol-cetostearyl ether 20 and mixtures of the aforementioned compounds. More preferably, the polyoxyethylene ether is a compound selected from the group of ceteareth-25, macrogol cetostearyl ether 20 and mixtures of the aforementioned compounds.

The term “polyglycerol ethers of fatty alcohols” refers to a compound of the formula R⁶O—(C₃H₆O₂)ₙ—H, where R⁶ is a branched or linear C₆-C₂₈ alkyl or C₆-C₂₈ alkenyl, and n is an integer greater than 1, preferably an integer from 2 to 10. It is preferable for the composition to contain 0.01 to 15.0 wt. %, 0.1 to 10.0 wt. %, or 1 to 5.0 wt. % polyglycerol ether.

The term “polyglycerol esters of fatty acids” refers to compounds containing both a polyglycerol unit and at least one C₆-C₂₆ alkyl or C₆-C₂₆ alkenyl carboxylic acid unit. These compounds may have the formula R⁷-R⁸—(C₃H₆O₂)ₙ—H, where R⁷ is a C₆-C₂₆ alkanoate group or C₆-C₂₆ alkenoate group, and R⁸ is a suitable linker molecule or a direct bond. Thus, the polyglycerol unit and the C₆-C₂₆ alkyl or C₆-C₂₆ alkenyl carboxylic acid unit may be directly linked by an ester bond or may contain a linking unit connecting these two units together. Non-limiting examples of this group are polyglyceryl-3-methylglucose distearate, polyglycerol polycrinoleate, polyglyceryl dimerat isostearate, polyglyceryl-2-laurate, polyglyceryl-2-sesquiisostearate, polyglyceryl-3-distearate (Cremophor GS 32), polyglyceryl-3-oleate, polyglyceryl-3-methylglycose distearate, polyglyceryl-4-caprate (polyglycerol caprate T2010190), polyglyceryl-4-diisostearate/polyhydroxystearate/sebacate (Isolan GPS) and polyglyceryl-4-isostearate.

In one embodiment, the surfactant comprises a cationic surfactant, such as a quaternary surfactant. Quaternary surfactants contain at least one N atom covalently bonded to 4 alkyl or aryl groups. This leads to a positive charge, regardless of the pH value. Alkyl betaine, alkylamidopropyl betaine and alkylamidopropyl hydroxysulfaine are advantageous. The cationic surfactants used according to the invention can also preferably be selected from the group of quaternary ammonium compounds, in particular benzyltrialkyl ammonium chlorides or bromides, such as benzyldimethylstearyl ammonium chloride, furthermore alkyltrialkyl ammonium salts, for example cetyltrimethyl ammonium chloride or bromide, alkyldimethylhydroxyethyl ammonium chlorides or bromides, dialkyldimethyl ammonium chlorides or bromides, alkylamideethyltrimethyl ammonium ether sulfates, alkylpyridinium salts, for example lauryl- or cetylpyrimidinium chloride, imidazoline derivatives and compounds having cationic character, such as amine oxides, for example alkyl dimethyl amine oxides or alkyl amino ethyl dimethyl amine oxides. It is particularly advantageous to use cetyltrimethyl ammonium salts.

In a preferred embodiment, the composition according to the invention is an oil-in-water emulsion (O/W) in which the surfactant components described above (also referred to as emulsifier components) have an HLB value of 8-40, preferably 8-22, particularly preferably 8-18. In another preferred embodiment, the composition according to the invention is a water-in-oil emulsion (W/O) in which the surfactant components described above (also referred to as emulsifier components) have an HLB value of 3-8, preferably 4-5. The calculation method for the combined HLB value is known and can be referred to as the Griffin method or Davies method (Ullmann's Encyclopedia of Industrial Chemistry, Part 9, Emulsion, 2005; Surfactant Application Encyclopedia, Liu Cheng, Beijing Industry Press, 1997).

In a further embodiment, the composition according to the invention contains at least one additive. Additives that are usually used in emulsions for treating the skin are preferred. The at least one additive may be present in a proportion of 0.01 wt. % to 12.0 wt. %, more preferably 0.25 wt. % to 10.0 wt. %, in particular 1.0 to 7.0 wt. %.

The at least one additive can further be selected from the group consisting of refatting agents, preservatives, stabilizers, fragrances, antioxidants, rheology modifiers, thickeners, care agents, dyes, pearlizing agents, brightening agents, solvents, sunscreens and combinations thereof.

Refatting agents, also called refatteners or superfatting agents, are lipophilic substances that can prevent a disruptive effect on the epidermal barrier function. Examples of refatting agents are wool fat, squalene, liquid paraffin, vegetable oils, silicones and cetyl palmitate.

Preservatives are substances used to preserve the composition by killing and/or inhibiting the growth of microorganisms that decompose it. Preferably, the preservatives can be selected from the group consisting of benzoic acid, benzoic acid derivatives, sorbic acid, sorbic acid derivatives, salicylic acid, salicylic acid derivatives, phenoxyethanol, parabens and combinations thereof. In a preferred embodiment, sodium benzoate and/or potassium sorbate are used as preservatives in the composition according to the invention. In a slightly acidic environment, sodium benzoate releases benzoic acid, and potassium sorbate releases sorbic acid. Both acids are said to have an antimicrobial effect.

Stabilizers can protect light-sensitive components from radiation and are preferably UV absorbers, such as benzophenone derivatives.

The addition of fragrances can provide a pleasant smell for the composition. Examples are the perfumes known to a person skilled in the art.

An antioxidant or antioxidant agent is a chemical compound that slows down or completely prevents oxidation of other components in the composition according to the invention. Possible antioxidants include, for example, citric acid, ascorbic acid and butylhydroxyanisole.

Rheology modifiers and thickeners can help to improve the application properties of the composition according to the invention. The addition of table salt (sodium chloride) can be considered as a rheology modifier and thickener. By adding table salt, the flowability of the composition according to the invention can be influenced within certain limits and adjusted to the required level. Naturally occurring thickeners can also be used as gellants, preferably selected from agar, xanthan gum, cellulose and/or cellulose derivatives or alginic acid.

In the context of the application, care products are to be understood to mean substances that care for the skin. Hydrolyzed wheat protein and allantoin have a skin-caring effect. Hydrolyzed wheat protein has mainly moisturizing properties.

Dyes are optionally used to give the composition according to the invention a characteristic color, such that it can be easily distinguished from other products.

The solvent used may be a solvent or solvent mixture popular with a person skilled in the art. Preferred solvents are ethanol or butylene glycol, especially 1,4-butylene glycol, propylene glycol and isopropyl alcohol. Ethanol is preferred. These solvents may preferably be present in the composition according to the invention in an amount of 0.1 to 70 wt. %. They are very particularly preferably contained in an amount of 0.1 to less than 5.0 wt. %. In addition, solvents help with the penetration of the active ingredients and thus increase their effectiveness. Suitable sunscreens are UVA filter substances and/or UVB filter substances and/or inorganic pigments.

Emulsions according to the invention can advantageously contain substances which absorb UV radiation in the UVB range, wherein the total amount of filter substances is, for example, 0.1 wt. % to 30 wt. %, preferably 0.5 to 10 wt. %, in particular 1 to 6 wt. %, based on the total weight of the emulsion.

The UVB filters can be oil-soluble or water-soluble. The following oil-soluble substances may be mentioned, for example:
3-benzylidene camphor and derivatives thereof, e.g., 3-(4-methylbenzylidene) camphor;
4-aminobenzoic acid derivatives, preferably 4-(dimethylamino)benzoic acid (2-ethylhexyl) ester, 4-(dimethylamino)benzoic acid amyl ester;
Esters of cinnamic acid, preferably 4-methoxycinnamic acid (2-ethylhexyl) ester, 4-methoxycinnamic acid isopentyl ester;
Esters of salicylic acid, preferably salicylic acid (2-ethylhexyl) ester, salicylic acid (4-isopropylbenzyl) ester, salicylic acid homomenthyl ester;
Derivatives of benzophenone, preferably 2-hydroxy-4-methoxybenzophenone, 2-hydroxy-4-methoxy-4′-methylbenzophenone, 2,2′-dihydroxy-4-methoxybenzophenone;
Esters of benzalmalonic acid, preferably 4-methoxybenzalmalonic acid di(2-ethylhexyl) ester; 2,4,6-trianilino-(p-carbo-2′-ethyl-1-hexyloxy)-1,3,5-triazine.

The following are advantageous water-soluble substances:
2-phenylbenzimidazole-5-sulfonic acid and the salts thereof, e.g., sodium, potassium or triethanol ammonium salts,
   Sulfonic acid derivatives of benzophenones, preferably 2-hydroxy-4-methoxybenzophenone-5-sulfonic acid and the salts thereof;
   Sulfonic acid derivatives of 3-benzylidene camphor, such as 4-(oxo-3-bornylidenemethyl)benzene sulfonic acid, 2-methyl-5-(2-oxo-3-bornylidenemethyl) sulfonic acid and the salts thereof.

The emulsions according to the invention can also contain UVA filters. Such filter substances are preferably derivatives of dibenzoylmethane, in particular 1-(4′-tert-butylphenyl)-3-(4′-methoxyphenyl)-propane-1,3-dione and 1-phenyl-3-(4-isopropylphenyl) propane-1,3-dione. The invention also relates to emulsions containing combinations of the aforementioned filters. The same amounts mentioned for UVB filter substances can be used for UVA filter substances. The emulsions according to the invention can also contain inorganic pigments which are commonly used in cosmetics to protect the skin from UV rays. These are oxides of titanium, zinc, iron, zirconium, silicon, manganese, aluminum, cerium and mixtures thereof. Particularly preferred are pigments based on titanium dioxide. The quantities mentioned for the above combinations can be used.

According to the invention, the composition is applied topically. Topical application means external application, in particular local, external application. Compositions according to the invention are preferably skin care products (i.e., treatment agents such as cures, lotions, shower gels, day or night creams, face creams, face fluids, face masks, skin protection creams, cleaning agents, sun protection lotions, deodorizing compositions, shaving creams, after shave balms, and/or a tonic), and not (purely) styling products. In other words, the topical compositions of the invention focus on the medical/pharmaceutical or cosmetic treatment of the skin.

The present invention further relates to the use of the composition according to the invention for treating the skin. According to the invention, the skin is particularly preferably the skin of the body, including the upper body and the lower body, and also including the hands, arms, legs and feet, and/or the skin of the face, including the neck and the nape. The skin preferably currently does not include the scalp, i.e., the skin beneath the hair.

In a preferred embodiment, the treatment of the skin relates to purely visual-aesthetic improvement, such as the creation of a smoother and simply better complexion. According to the invention, however, additional cosmetic/medical effects are also particularly preferably achieved, such as an improved protective function and a strengthened barrier property. In addition, wound healing is improved.

In other words, the use according to the invention leads to a significant improvement in the epidermal barrier functions and the epidermal barrier integrity, to the improvement in the appearance of the skin, and to the increase in the moisture content of the skin. This is accompanied by an increase in the cohesion of the stratum corneum and the homeostasis of the skin barrier, and ultimately results in improved protection against infections (microbial diseases).

When used medically, the invention encompasses both the therapeutic and the prophylactic treatment of skin diseases. The diseases are preferably microbial skin infections, skin irritations, rough skin, dry skin, skin irritations, itching, pruritus, allergies, psoriasis, psoriatic arthritis, eczema, scleroderma, atopic dermatitis, contact dermatitis, systemic lupus erythematosus, acne and susceptibility to contact allergies.

The non-therapeutic (i.e., purely cosmetic) use includes in particular the treatment of cosmetic indications of the skin, in particular selected from rough skin, dry skin, skin irritations, itching and pruritus, as well as the prevention of skin infections and the reduction in susceptibility to contact allergies.

The emulsions according to the invention are particularly effective at improving epidermal barrier functions and epidermal barrier integrity, in promoting wound healing, in treating and/or preventing inflammatory conditions of the skin, in treating and/or preventing disorders associated with the lack of regeneration of the epidermis, and in improving the appearance of the skin, increasing the moisture content of the skin, promoting natural skin protection and/or revitalizing the skin.

The invention shall be illustrated by the following compositions, without, however, wishing to restrict it to the specific examples.

### Experimental Part

The following compositions were prepared by homogenization measures known to those skilled in the art. The amount of each of the components was chosen so that their weight proportion corresponds to weight proportions specified in the finished composition.

### EXAMPLE 1

Composition in the form of an emulsion containing the following components:

| | |
|---|---|
| Zinc stearate | 1.25 wt. % |
| Magnesium sulfate | 0.5 wt. % |
| Sorbitan isostearate & polyglyceryl-3 | 4.5 wt. % |
| polyricinoleate | |
| Hexyldecanol & hexyldecyl laurate | 25.0 wt. % |
| Glycerin | 2.0 wt. % |
| Citric acid | 0.4 wt. % |
| Sodium dimethyl glycinate | 0.4 wt. % |
| Perfume | 0.2 wt. % |
| Preservative | q.s. |
| Water | up to 100 wt. % |

### EXAMPLE 2

Composition in the form of an emulsion containing the following components:

| | |
|---|---|
| Cetearyl ethylhexanoate | 20.0 wt. % |
| Carbomer | 0.2 wt. % |
| Polyglyceryl-3 methylglucose distearate | 3.0 wt. % |
| Cetearyl alcohol & sodium cetearyl sulfate | 2.0 wt. % |
| Glyceryl stearate | 1.5 wt. % |
| Citric acid | 0.4 wt. % |
| Sodium dimethyl glycinate | 0.6 wt. % |
| Perfume | 0.2 wt. % |
| Preservative | q.s. |
| Water | up to 100 wt. % |

### EXAMPLE 3

Composition in the form of an emulsion (viscosity 7,500 mPa·s) containing the following components:

| | |
|---|---|
| Citric acid | 0.83 wt. % |
| Sodium dimethyl glycinate | 1.0 wt. % |
| Phenoxyethanol | 1.00 wt. % |
| Caprylic/capric triglycerides | 20.00 wt. % |
| Steareth-2 | 3.00 wt. % |
| Steareth-21 | 3.00 wt. % |
| Xanthan gum | 2.00 wt. % |
| Water | up to 100 wt. % |

### EXAMPLE 4

Composition in the form of an emulsion (viscosity 32,000 mPa·s) containing the following components:

| | |
|---|---|
| Citric acid | 0.85 wt. % |
| Sodium dimethyl glycinate | 1.0 wt. % |
| Phenoxyethanol | 1.0 wt. % |
| Caprylic/capric triglycerides | 14.0 wt. % |
| Steareth-2 | 3.0 wt. % |
| Steareth-21 | 3.0 wt. % |
| Xanthan gum | 1.0 wt. % |
| Cetearyl alcohol | 6.0 wt. % |
| Water | up to 100 wt. % |

### EXAMPLE 5

Composition in the form of an emulsion containing the following components:

| | |
|---|---|
| Caprylic/capric triglycerides | 20 wt. % |
| Steareth-2 | 3 wt. % |
| Steareth-21 | 3 wt. % |
| Xanthan gum | 2 wt. % |
| Sodium dimethyl glycinate | 1 wt. % |
| Citric acid | 0.8 wt. % |
| Perfume Oil | q.s. |
| Preservative | q.s. |
| Water | up to 100 wt. % |

### EXAMPLE 6

Composition in the form of a gel containing the following components:

| | |
|---|---|
| Sodium dimethyl glycinate | 1.0 wt. % |
| Anhydrous citric acid | 0.83 wt. % |
| Ethanol | 10.0 wt. % |
| Poloxamer 407 | 18.0 wt. % |
| Water | up to 100 wt. % |

### EXAMPLE 7

Composition in the form of an emulsion containing the following components:

| | |
|---|---|
| Caprylic/capric triglycerides | 20 wt. % |
| Cetyl alcohol | 2.7 wt. % |
| Polyglyceryl-3 methylglucose distearate | 3.5 wt. % |
| Sodium cetearyl sulfate | 0.3 wt. % |
| Sodium dimethyl glycinate | 1.0 wt. % |
| Citric acid | 0.7 wt. % |
| Perfume Oil | q.s. |
| Preservative | q.s. |
| Water | up to 100 wt. % |

### REFERENCE EXAMPLE 1

Composition in the form of an emulsion (with inorganic acid) containing the following components:

| | |
|---|---|
| Caprylic/capric triglycerides | 20 wt. % |
| Cetyl alcohol | 2.7 wt. % |
| Polyglyceryl-3 methylglucose distearate | 3.5 wt. % |
| Sodium cetearyl sulfate | 0.3 wt. % |
| Sodium dimethyl glycinate | 1.0 wt. % |
| Hydrochloric acid | 0.7 wt. % |
| Perfume Oil | q.s. |
| Preservative | q.s. |
| Water | up to 100 wt. % |

### REFERENCE EXAMPLE 2

Composition in the form of an emulsion (without Na-dimethylglycinate) containing the following components:

| | |
|---|---|
| Caprylic/capric triglycerides | 20 wt. % |
| Cetyl alcohol | 2.7 wt. % |
| Polyglyceryl-3 methylglucose distearate | 3.5 wt. % |
| Sodium cetearyl sulfates | 0.3 wt. % |
| Citric acid | 0.7 wt. % |
| Perfume oil | q.s. |
| Preservative | q.s. |
| Water | up to 100 wt. % |

### REFERENCE EXAMPLE 3

Composition in the form of an emulsion (without Na-dimethylglycinate) containing the following components:

| | |
|---|---|
| Water | up to 100 wt. % |
| Citric acid | 0.83 wt. % |
| 25% Sodium hydroxide solution | 1.35 wt. % |
| Phenoxyethanol | 1.00 wt. % |
| Caprylic/capric triglycerides | 14.0 wt. % |
| Steareth-2 | 3.0 wt. % |
| Steareth-21 | 3.0 wt. % |
| Xanthan gum | 1.0 wt. % |
| Cetylstearyl alcohol | 6.0 wt. % |

### REFERENCE EXAMPLE 4

Composition in the form of an emulsion (without Na-dimethylglycinate) containing the following components:

| | |
|---|---|
| Caprylic/capric triglycerides | 20 wt. % |
| Steareth-2 | 3 wt. % |
| Steareth-21 | 3 wt. % |
| Xanthan gum | 2 wt. % |
| Citric acid | 0.8 wt. % |
| Perfume oil | q.s. |
| Preservative | q.s. |
| Water | up to 100 wt. % |

### Study Design and Results

1. As part of an in-vitro study, the effect of Na-dimethylglycinate was investigated in a cell culture model with human horn-forming keratinocyte cells. For this purpose, HaCaT cells were cultured for 1, 3, 5, and 7 days in DMEM medium (including fetal calf serum and an antibiotic-antimycotic mix) and, among other things, the viability, proliferation and migration of the cells were determined using suitable measuring methods, as well as the expression of the growth factors relevant for cell growth.

### Proof of Viability:

For this measurement, a so-called MTT assay was used to determine cellular metabolic activity as an indicator of cell viability and cytotoxicity. This colorimetric assay is based on the reduction of a yellow tetrazolium salt (3-(4,5-dimethylthiazol-2-yl)-2,5-diphenyltetrazolium bromide or MTT) to violet formazan crystals by metabolically active cells.

Human epidermal keratinocyte cells (HaCaT) were seeded in a 96-well plate at a cell density of 5,000 cells/well and cultured in medium (DMEM with 10% FBS, 1% penicillin-streptomycin, 0.5% Fungizone). The next day and after a further 24 h, 48 h and 72 h of cultivation at 37° C. and 5 vol. % CO₂, the cell culture medium was changed without (control) or with the active ingredient concentrations of sodium dimethylglycinate (DMG) already contained. The measurement was carried out analogously to previously published studies in B. I. Tóth, N. Dobrosi, A. Dajnoki, G. Czifra, A. Oláh, A. G. Szöllösi, I. Juhász, K. Sugawara, R. Paus, T. Bíró, J Invest Dermatol 2011, 131, 1,095-1,104.

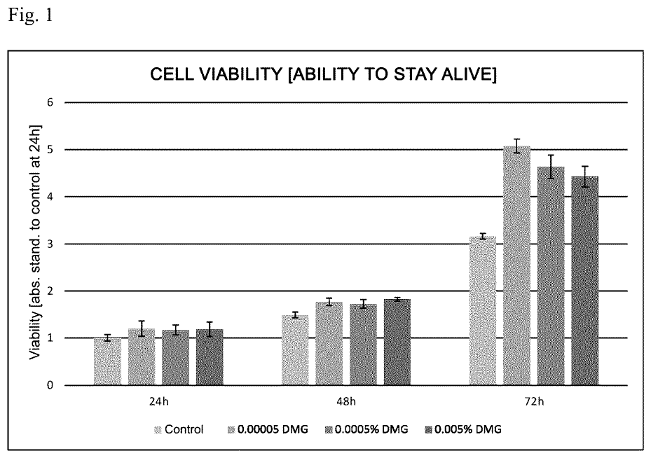

FIG. 1 shows the cell viability after 24 h, 48 h and 72 h of cultivation, and a significant increase can be seen as a result of the addition of DMG after 48 h and 72 h. Cell viability was determined using MTT assay. The absorbance was measured using quadruple determination and standardized to the control at 24 h. The mean value with standard deviation is given.

### Evidence of Proliferation:

To measure proliferation, a so-called CyQUANT assay was performed. In this fluorescence-based assay, the fluorescent dye used binds to DNA (deoxyribonucleic acid), whereby the content of cellular DNA is a direct measure of the number of cells within a sample. Human epidermal keratinocyte cells (HaCaT) were seeded in a 96-well plate at a cell density of 5,000 cells/well and cultured in medium (DMEM with 10% FBS, 1% penicillin-streptomycin, 0.5% Fungizone). The next day and after a further 24 h, 48 h and 72 h of cultivation at 37° C. and 5 vol. % CO₂, the cell culture medium was changed without (control) or with the active ingredient concentrations already contained. The measurements were performed analogously to previously published studies in A. Oláh, B. I. Tóth, I. Borbíró, K. Sugawara, A. G. Szöllösi, G. Czifra, B. Pál, L. Ambrus, J. Kloepper, E. Camera, The Journal of clinical investigation 2014, 124, 3,713-3,724.

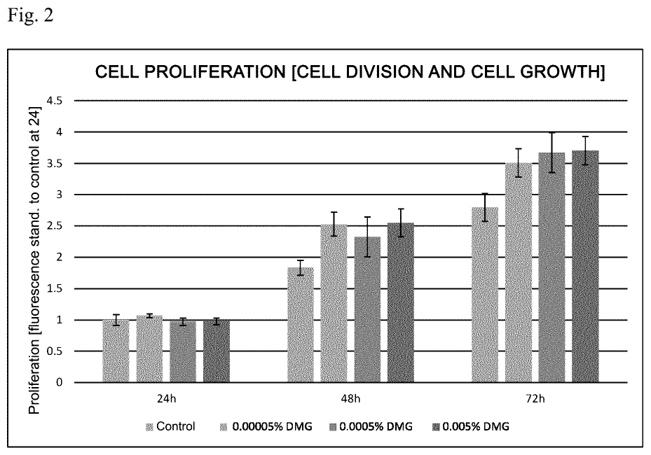

FIG. 2 shows cell proliferation after 24 h, 48 h and 72 h of cultivation, and a significant increase can be seen as a result of the addition of DMG after 48 h and 72 h. Cell proliferation was determined using the CyQUANT assay. Fluorescence was measured using triple determination each time and standardized to the control at 24 h. The mean value with standard deviation is given.

### Proof of Migration:

To measure migration, a so-called wound healing assay was performed, based on previously published studies in T. Kawabata, T. Otsuka, K. Fujita, G. Sakai, R. Matsushima-Nishiwaki, O. Kozawa, H. Tokuda, International journal of molecular medicine 2018, 42, 3,149-3,156. The principle is based on measuring the migration of cells over time on a cultivation surface yet to be colonized. For this purpose, 20,000 cells were seeded into two adjacent wells or cavities separated by a silicone insert (having a standardized width) or cultured in medium (DMEM with 5% FBS, 1% penicillin-streptomycin, 0.5% Fungizone) for 48 h at 37° C. and 5 vol. % CO₂. Subsequently, the plastic insert was removed (“creation of the wound”), and the migration of the cells was documented by determining the cultivation surface not populated by cells over time using image recordings. At the same time, the cell culture medium was changed without (control) or with the active ingredient concentrations of DMG already contained immediately after removal of the plastic insert (0 h) and after a further 24 h. The evaluation of the images and the determination of the cultivation surface not populated by cells was carried out using specific software (ImageJ).

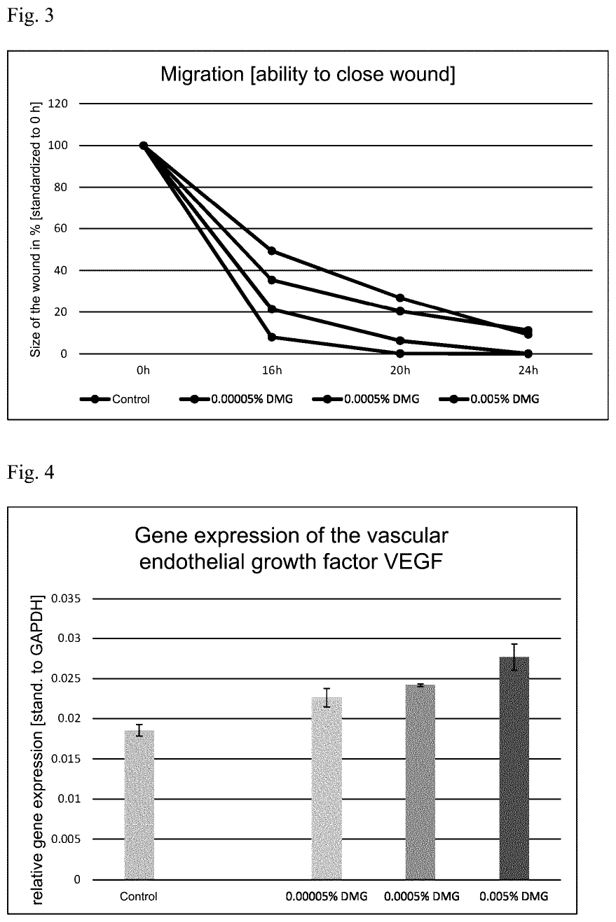

FIG. 3 shows the migration over time and using different DMG concentrations. Migration and wound closure occur significantly faster with DMG compared to the control without DMG. At time 0 h, the wound was created and images were taken after 16 h, 20 h, and 24 h of cultivation. Based on the images, the cultivation surface, which had not yet been populated by cells, was determined using the ImageJ software. The measured values are standardized to the time point 0 h (maximum size of the wound) and given in %.

### Detection of VEGF Gene Expression:

VEGF (vascular endothelial growth factor) promotes the growth and formation of new blood and lymph vessels. Gene expression was measured using a standard method of quantitative real-time PCR (qRT-PCR).

Human epidermal keratinocyte cells (HaCaT) were seeded in a 6-well plate at a cell density of 140,000 cells/well and cultured in medium (DMEM with 10% FBS, 1% penicillin-streptomycin, 0.5% Fungizone) at 37° C. and 5 vol. % CO₂. The next day, the cell culture medium was changed without (control) and with the active ingredient concentrations already contained and the cells were harvested after 24 h. Gene expression was measured using qRT-PCR based on previously published studies in B. V. Diaz, M.-C. Lenoir, A. Ladoux, C. Frelin, M. Demarchez, S. Michel, Journal of Biological Chemistry 2000, 275, 642-650.

FIG. 4 shows the gene expression of VEGF after 24 h of cultivation, and a significant increase in gene expression can be seen as a result of the addition of DMG. Shown is the relative gene expression of VEGF determined by qRT-PCR (triple determination, standardized to the corresponding gene expression of a constitutively expressed gene; GAPDH-glyceraldehyde-3-phosphate dehydrogenase). The mean value with standard deviation is given.

Surprisingly, it was shown that DMG had a positive effect on the growth-relevant parameters of HaCaT cells and that the expression of the growth factor VEGF was significantly increased compared to the treatment of HaCaT cells without DMG.

2. The cream according to the invention from Example 7 as well as the skin cream from Reference Example 1 (with inorganic acid) and Reference Example 2 (without Na-dimethylglycinate) were applied to 4 women and men (phototype Fitzpatrick I-III, age: 20-64 years) as part of a controlled and blinded skin moisture measurement after a single application to the forearm. Before product application, 4 randomized areas on the forearm were defined, and baseline skin moisture values were measured (using Comeometer, Derma Unit SC 2 from Courage+Khazaka Electronic GmbH, Germany, at 22° C. room temperature). An amount of approximately 2 mg/cm² of the respective product was applied to the corresponding area on the forearm and massaged in for approximately 30 seconds, with one area remaining untreated as a control field. Skin moisture was measured after 30, 60, 120 and 180 minutes.

In comparison with the two reference examples and the untreated control field, it was shown that skin moisture was significantly increased by applying the cream according to the invention.

3. The cream according to the invention from Example 7 as well as the skin cream from Reference Example 1 (with inorganic acid) were tested by osmolality measurement. Osmolality is a measure of the total number of dissolved chemical units and thus indicates the value of the osmotic pressure of a formulation. The osmolality is measured by determining the freezing point depression (using an automatic semi-micro osmometer from KNAUER Wissenschaftliche Geräthe GmbH, Germany) according to established test procedures according to Ph. Eur 8.0, 2.2.35 and is expressed in mosmol/kg.

It was shown that the osmolality and the associated osmotic pressure of the formulation in the cream according to the invention is significantly increased compared to the reference example.

4. The cream according to the invention from Example 3 and the skin cream from Example 4 were analyzed as part of formulation release tests (“in vitro release testing”). The release of Na-dimethylglycinate from the different formulations was determined using a Phoenix DB-6 diffusion cell and using established procedures (see, among others, Mckinney R D, Dunbar J R. In Vitro Release Tests of Ketoprofen from Pluronic Lecithin Organogel versus Lipoderm Using Immersion Cells and the Phoenix DB-6 Dry Heat Diffusion Tester. Int J Pharm Compd. 2021 May-Jun; 25 (3): 241-245.)

To carry out the test, a polyether sulfone membrane (pore size 0.45 μm, diameter 25 mm) was clamped in the diffusion cell, and a 0.2 M phosphate buffer solution (pH 2.5) was used as the receptor medium at 32° C. and a 400 rpm stirring speed. 500 μL of the corresponding product were applied to the membrane, and, after 30, 60, 120 and 180 minutes, samples were taken from the receptor and the removed volume was filled with fresh receptor medium. The content of Na-dimethylglycinate in the samples was determined using the HPLC (“high pressure liquid chromatography”) method. It was shown that the release of Na-dimethylglycinate from the cream according to the invention with a viscosity of 7,500 mPa·s is significantly faster and more effective than, for example, with a higher viscosity of 32,000 mPa·s.

5. The cream according to the invention from Example 3 and the skin cream from Example 4 were analyzed in penetration tests on human skin biopsies. The penetration of Na-dimethylglycinate from the different formulations was carried out using a Franz diffusion cell (Crown Glass Company INC, Somerville, New York, USA). In principle, the Franz cell consists of a donor and an acceptor chamber that are separated from one another by a skin biopsy and represents an established system for testing skin penetration (see, among others, Trauer S, Patzelt A, Otberg N, Knorr F, Rozycki C, Balizs G, Buttemeyer R, Linscheid M, Liebsch M, Lademann J., Permeation of topically applied caffeine through human skin—a comparison of in vivo and in vitro data. Br J Clin Pharmacol. 2009 August; 68 (2): 181-6). While carrying out the test, the apparatus was kept at 32° C., and distilled water was used as the receptor medium. 20 mg of test product were applied to the skin biopsy (topical or external side of the biopsy) in the donor chamber, and skin punches were removed after 1, 6 and 24 hours. To determine the content of Na-dimethylglycinate, the skin punches were processed according to established methods, and the content was determined in the individual skin layers and in the receptor medium using the HPLC (“high pressure liquid chromatography”) method.

It was shown that the penetration of Na-dimethylglycinate into and through the skin from the cream according to the invention that has a viscosity of 7,500 mPa·s is significantly faster and more effective than, for example, with a higher viscosity of 32,000 mPa·s.

6. The cream according to the invention from Example 5 and the skin cream from Reference Example 4 were applied to the face of 20 women (phototype Fitzpatrick I-III, age: 20-64 years) in a controlled, blinded half-face test. An amount of 80 μL (controlled volume measurement via Gilson Pipetman Microman E, M250E, 50-250 microliters, 7-4305) was applied simultaneously to one half of each test subject's face and evenly distributed. After one minute (the cream being absorbed into the skin), parameters relating to the effectiveness of the cream were determined subjectively by the test subjects and objectively by the evaluation of a trained and experienced beautician. The parameters evaluated included, for example: skin feel, evenness of complexion, skin moisture.

It was shown that the test subjects themselves felt the skin was much more cared for and softer as a result of applying the cream according to the invention compared to the reference example. At the same time, the skin's appearance was objectively assessed as much more even after applying the test cream compared to the reference example.

1. A topical composition containing
a) dimethylglycine and/or a salt of dimethylglycine,
b) at least one carboxylic acid,
wherein the weight ratio of a) to b) ranges from 1:0.1 to 1:10, preferably from 1:0.7 to 1:1.5.

2. The composition according to claim 1, wherein the composition contains 0.00001 to 25.0 wt. % dimethylglycine and/or a salt of dimethylglycine.

3. The composition according to claim 1 and/or claim 2, wherein the carboxylic acid is selected from the group consisting of adipic acid, ascorbic acid, lactic acid, acetic acid, propanoic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methane sulfonic acid, ethane sulfonic acid, p-toluene sulfonic acid, salicylic acid, azelaic acid, cinnamic acid, formic acid, (meth)acrylic acid, nicotinic acid and mixtures thereof.

4. The composition according to any one of the preceding claims, wherein the composition contains surfactants in an amount of 2 to 40 wt. %.

5. The composition according to any one of the preceding claims, wherein the composition contains at least one further active ingredient selected from caffeine, menthol, biotin, zinc PCA, niacinamide, panthenol, ectoine, ubiquinone-10, taurine, pantolactone, echinacea, carnitine, tocopheryl acetate and combinations thereof.

6. The composition according to any one of the preceding claims, wherein the composition contains at least one additive selected from emulsifiers, refatting agents, preservatives, stabilizers, fragrances, antioxidants, rheology modifiers, thickeners, care agents, dyes, pearlizing agents, brightening agents, solvents, sunscreens and combinations thereof.

7. The composition according to any one of the preceding claims, wherein the composition is a care composition, preferably for promoting the metabolism of the skin.

8. A use of the composition according to any one of the preceding claims for treating the skin.

9. The use according to claim 8, wherein the skin is treated in order to improve the epidermal barrier functions and the epidermal barrier integrity, to promote wound healing, to treat and/or prevent inflammatory conditions of the skin, to treat and/or prevent disorders associated with inadequate regeneration of the epidermis, and to improve the appearance of the skin, to increase the moisture content of the skin, to promote natural skin protection and/or to revitalize the skin.

10. The use according to claims 8 and/or 9, wherein the use is a cosmetic or medicinal use.
